(19) United States
(12) Patent Application Publication
(10) Pub. No.: US 2014/0377773 A1
(43) Pub. Date: Dec. 25, 2014

(54) Detection Marker for Anticancer Effects by Selenomethionine as an Inhibitor of Environmental Toxicity

(71) Applicant: Dongguk University Industry-Academic Cooperation Foundation, Seoul (KR)

(72) Inventors: Young-Rok Seo, Seoul (KR); Md. Mujibur Rahman, Seoul (KR); Jong-Il Weon, Seoul (KR); Ju Han Lee, Busan (KR); Jee Young Kwon, Seoul (KR); Hye Lim Kim, Seoul (KR)

(73) Assignee: Dongguk University Industry-Academic Cooperation Foundation, Seoul (KR)

(21) Appl. No.: 13/954,053

(22) Filed: Jul. 30, 2013

(30) Foreign Application Priority Data
Jun. 21, 2013 (KR) 10-2013-0071632

(51) Int. Cl. G01N 33/574

(52) U.S. Cl. CPC G01N 33/57419
USPC 435/7.4; 435/7.9

(57) ABSTRACT

The present invention relates to specific markers capable of detecting the development of colorectal cancer and the colorectal cancer inhibitory effect of SeMet (selenomethionine) having a chemopreventive effect against colorectal cancer. When the expressions of the biomarkers according to the present invention are measured and the expression levels thereof are analyzed in combination, whether SeMet (selenomethionine) is to be administered to prevent colorectal cancer can be determined and the development of colorectal cancer and the inhibitory effect of SeMet (selenomethionine) against the development of colorectal cancer can be monitored. Thus, these markers can be effectively used to observe the colorectal cancer inhibitory effect of SeMet (selenomethionine) and the prognosis of colorectal cancer resulting from the intake of SeMet (selenomethionine).

### CROSS-REFERENCE TO RELATED APPLICATIONS

This application claims priority to and the benefit of Korean Patent Application No. 10-2013-0071632, filed on Jun. 21, 2013, which is incorporated herein by reference in its entirety.

### SEQUENCE LISTING

Incorporated by reference herein in its entirety is the Sequence Listing entitled “Sequence_Listing_ST25,” created Jul. 30, 2013, size of 9.97 kilobytes.

The patent or application file contains at least one drawing executed in color. Copies of this patent or patent application publication with color drawing(s) will be provided by the Office upon request and payment of the necessary fee.

### BACKGROUND OF THE INVENTION

1. Field of the Invention

The present invention relates to specific markers capable of detecting the development of colorectal cancer and the colorectal cancer inhibitory effect of SeMet (selenomethionine) having a chemopreventive effect against colorectal cancer.

2. Description of the Prior Art

Colorectal cancer (CRC) is a disease that affects 1.2 million people worldwide per year and causes 608,700 deaths (year 2008) worldwide. Colorectal cancer accounts for about 8% of mortality caused by all cancers and has a high incidence rate in Australia, New Zealand, Europe and North America. Pathologically, CRC results from the conversion of normal colorectal endothelial cells into adenomatous polyps and finally into invasive cancer and requires several progression stages and developmental stages. Colorectal cancer is caused mainly by genetic and environmental factors, and the major risk factors of colorectal cancer include smoking, physical inactivity, obesity, intake of red meats and processed meats, and excessive intake of alcohol. Chemical substances are used to minimize the above-described risk factors and to reduce the initiation of carcinogenic processes or allow such processes to retrogress.

It is known that regular intake of selenium as a supplement inhibits tumorogenesis and reduces the risk of carcinogenesis (Tinggi, U. (2008). Environ Health Prey Med, 13, 102-8.). It was found that SeMet (Selenomethionine) hylselenocysteine, methaneselenenic acid or methaneseleninic acid that is a methylated form of selenium may have a defense effect against the progression of tumors (Brigelius-Flohe, R. (2008). Chem Biodivers, 5, 389-95). Inorganic selenium shows cytotoxicity, unlike selenomethionine that is organic selenium. It is known that selenium and selenium-containing compounds act similar to antioxidants that show chemopreventive effects. Recent studies on the pre-appearance of symptoms, epidemiological studies and clinical trials revealed that selenium is a potent candidate for chemoprevention (Nelson, M. A., et al. (2005). Tumor Progression and Therapeutic Resistance, 1059, 26-32). It is believed that methylselenol and related metabolites target both endothelial and colon cancer cells and play an important role in chemoprevention, and the risk of CRC in patients who take selenium was reduced by about 50% (Marshall, J. R. (2008). Gastroenterol Clin North Am, 37, 73-82, vi.).

Previous studies indicated that selenomethionine reduces the development of AOM-induced premalignant lesions through a polyamine-independent mechanism in AOM-DSS mouse models (Baines, A. T., et al. (2000). Cancer Lett, 160, 193-8.). Thus, it will be significant from a viewpoint of treatment and prognosis to identify molecules that induce SeMet (selenomethionine)-mediated chemoprevention against CRC.

Accordingly, the present inventors have conducted studies on the chemopreventive effect of SeMet (selenomethionine) against the development of adenomatous polyps in AOM-DSS mice, and as a result, have found that biomarkers associated with SeMet (selenomethionine)-mediated inhibition of colorectal cancer were identified by proteomics analysis and that when the expression levels thereof are analyzed in combination, whether SeMet (selenomethionine) is to be administered can be determined and the development of colorectal cancer and the inhibitory effect of SeMet (selenomethionine) against the development of colorectal cancer can be monitored.

### SUMMARY OF THE INVENTION

An object of the present invention is to provide a composition and kit for detecting the colorectal cancer inhibitory effect of SeMet (selenomethionine), which are used to monitor the colorectal cancer inhibitory effect of SeMet (selenomethionine) by measuring the expression level of PHB (prohibitin), PNP (purine nucleoside phosphorylase), ANXA2 (annexin A2) and/or CRP (C-reactive protein) that is a biomarker of the present invention and to analyze the expression of the biomarker using an antibody specific to the biomarker.

To achieve the above object, the present invention provides a composition for detecting the colorectal cancer inhibitory effect of SeMet (selenomethionine).

The present invention also provides a kit for detecting the colorectal cancer inhibitory effect of SeMet (selenomethionine), the kit comprising the above composition.

The present invention also provides a method for providing information required to monitor the colorectal cancer inhibitory effect of SeMet (selenomethionine).

### BRIEF DESCRIPTION OF THE DRAWINGS

FIG. 1A shows the period of treatment with AOM, DSS and/or SeMet (selenomethionine) for each mouse group of the AOM-DSS model.

FIG. 1B shows the mouse groups of the AOM-DSS model.

FIG. 1C shows the colons of mouse groups of the AOM-DSS model.

FIG. 1D: shows the frequency of development of polyps and the size of polyps in the colons of mouse groups of the AOM-DSS model.

FIG. 2A shows the colon tissues of each mouse groups of the AOM-DSS model stained with hematoxylin and eosin (H & E).

FIG. 2B shows the results of analysis of 8-OHdG (8-hydroxy-2′-deoxyguanosine) in the colon tissues of each mouse groups of the AOM-DSS model.

FIG. 2C shows the expression of 8-OHdG in the colon tissues of each mouse groups of the AOM-DSS model.

FIG. 3A shows the expression of 76 proteins in group 3 treated with AOM-DSS alone.

FIG. 3B shows the expression of 76 proteins in group 4, pretreated with SeMet (selenomethionine) and treated with AOM-DSS.

FIG. 4 shows the results of analysis using Pathway Studio 8 software for the networks of 30 proteins that showed a difference in expression between group 3 treated with AOM-DSS alone and group 4, pretreated with SeMet (selenomethionine) and treated with AOM-DSS.

FIG. 5A shows the expressions of PHB, PNP, ANXA2 and CRP in the mouse groups of the AOM-DSS model.

FIG. 5B shows the expression levels of PHB, PNP, ANXA2 and CRP in the colon tissues of mouse groups of the AOM-DSS models.

FIG. 6 shows the results of Western blot analysis of the expressions of PHB, PNP, ANXA2 and CRP in the mouse groups of the AOM-DSS model.

FIG. 7 shows the results of analysis Pathway Studio 8 software for the networks of PHB, PNP, ANXA2 and CRP in the intracellular signaling pathway.

### DETAILED DESCRIPTION OF THE INVENTION

As used herein, the phrase “chemopreventive activity of SeMet (selenomethionine) against colorectal cancer” means that the development or progression of colorectal cancer is inhibited by the administration or intake of SeMet (selenomethionine).

As used herein, the term “AOM-DSS mouse model” refers to an animal model, which has colorectal cancer induced by treatment with AOM and DSS and is generally used in studies on the development of colorectal cancer (Tanaka, T., et al. (2003). Cancer Sci, 94, 965-73. 19. and Krehl, S., et al. (2012). Carcinogenesis, 33, 620-8.).

“PHB (prohibitin)” that is a marker of the present invention is a protein that regulates cell proliferation, apoptosis, transcription and mitochondrial protein folding and acts as a cell-surface receptor. It may have an amino acid sequence set forth in SEQ ID NO: 1.

“PNP (purine nucleoside phosphorylase)” that is a marker of the present invention is an enzyme that catalyzes a reaction which reversibly converts purine riboside to the corresponding nucleotide. It may have an amino acid sequence set forth in SEQ ID NO: 2.

“ANXA2 (annexin A2)” that is a marker of the present invention is a calcium and phospholipid-binding protein that plays an important role in signaling, cell differentiation and proliferation. It may have an amino acid sequence set forth in SEQ ID NO: 3.

“CRP (C-reactive protein)” that is a marker of the present invention is a protein very close to chronic inflammation. It may have an amino acid sequence set forth in SEQ ID NO: 4.

“8-OHdG (8-hydroxy-2′-deoxyguanosine)” that is a marker of the present invention is an oxidized DNA nucleotide that is used as an oxidative stress marker.

The proteins of the present invention may comprise a nucleotide sequence having a sequence homology of 70% or higher, preferably 80% or higher, more preferably 90% or higher, and most preferably 95% or higher, to the amino acid sequence of each of the proteins.

The percentage of sequence homology to the amino acid sequence is determined by comparing two optimally aligned sequences over a comparison region, wherein the portion of the amino acid sequence in the comparison region may comprise additions or deletions as compared to the reference sequence (that does not comprise additions or deletions) for optimal alignment of the two sequences.

The present invention provides a composition for detecting the colorectal cancer inhibitory effect of SeMet (selenomethionine), the composition comprising agents for measuring the expression levels of PHB (prohibitin) or PNP (purine nucleoside phosphorylase) protein and ANXA2 (annexin A2) or CRP(C-reactive protein) protein.

The agents for measuring the expression levels are preferably probes, primers, antibodies or aptamers. Any binding agents may be used without limitation in the present invention, as long as they can detect the expressions of PHB, PNP, ANXA2 and CRP that are the markers of the present invention.

The detection of the expressions of the proteins may be performed by biochip analysis, gel electrophoresis, radioactivity measurement, fluorescence measurement or phosphorescence measurement, but is not limited thereto.

Preferably, PHB (prohibitin) has the amino acid sequence set forth in SEQ ID NO: 1, PNP (purine nucleoside phosphorylase) has the amino acid sequence set forth in SEQ ID NO: 2, ANXA2 (annexin A2) has the amino acid sequence set forth in SEQ ID NO: 3, and CRP (C-reactive protein) has the amino acid sequence set forth in SEQ ID NO: 4, but are not limited thereto.

The present invention also provides a kit comprising the inventive composition for detecting the colorectal cancer inhibitory effect of SeMet (selenomethionine).

In addition to the inventive composition for detecting the colorectal cancer inhibitory effect of SeMet (selenomethionine), the kit of the present invention may further comprise expression reference tables for components or a control group, which make it easy to detect the expressions of the markers.

The present invention also provides a method for providing information required to monitor the colorectal cancer inhibitory effect of SeMet (selenomethionine), the method comprising a step of measuring the expression of at least one protein selected from the group consisting of PHB (prohibitin), PNP (purine nucleoside phosphorylase), ANXA2 (annexin A2) and CRP (C-reactive protein) in a sample separated from a subject.

The sample is preferably selected from the group consisting of tissue, phlegm, blood, plasma and urine, and the tissue is preferably colon tissue or a colon cell isolated therefrom, but is not limited thereto.

Preferably, PHB (prohibitin) has the amino acid sequence set forth in SEQ ID NO: 1, PNP (purine nucleoside phosphorylase) has the amino acid sequence set forth in SEQ ID NO: 2, ANXA2 (annexin A2) has the amino acid sequence set forth in SEQ ID NO: 3, and CRP (C-reactive protein) has the amino acid sequence set forth in SEQ ID NO: 4, but are not limited thereto.

The expression of PHB (prohibitin) or PNP (purine nucleoside phosphorylase) is preferably increased compared to a control group by administration of SeMet (selenomethionine), and the expression is decreased by the development of colorectal cancer. When the development of colorectal cancer was inhibited by SeMet (selenomethionine), the expression of PHB (prohibitin) or PNP (purine nucleoside phosphorylase) is preferably decreased by administration of SeMet (selenomethionine), but is increased compared to a control group. However, the scope of the present invention is not limited thereto.

The expression of ANXA2 (annexin A2) or CRP (C-reactive protein) is preferably increased compared to a control group by administration of colorectal cancer. When the development of colorectal cancer was inhibited by administration of SeMet (selenomethionine), the expression of ANXA2 (annexin A2) or CRP (C-reactive protein) decreases compared to when colorectal cancer develops. However, the scope of the present invention is not limited thereto.

In a preferred embodiment of the present invention, in monitoring of the colorectal cancer inhibitory effect of SeMet (selenomethionine), when the expression of PHB (prohibitin) or PNP(purine nucleoside phosphorylase) and the expression of ANXA2 (annexin A2) or CRP (C-reactive protein) increase together, it is determined that SeMet (selenomethionine) has a tumor preventive or inhibitory effect. In a more preferred embodiment of the present invention, when the expressions of PHB (prohibitin), PNP (purine nucleoside phosphorylase), ANXA2 (annexin A2) and CRP (C-reactive protein) increase together, it is determined that SeMet (selenomethionine) has a tumor preventive or inhibitory effect. However, the scope of the present invention is not limited thereto.

In a specific example of the present invention, the colorectal cancer inhibitory effect of SeMet (selenomethionine) was observed in a mouse model having colorectal cancer induced by AOM-DSS, and proteins whose expressions changed when the development of colorectal cancer was inhibited by SeMet (selenomethionine) were investigated, thereby PHB, PNP, ANXA2 and CRP proteins that target SeMet (selenomethionine). In addition, when SeMet (selenomethionine) was administered, the expressions of PHB and PNP were up-regulated, and the expressions of ANXA2 and CRP did not change. When colorectal cancer developed, the expressions of PHB and PNP were down-regulated, and the expression of ANXA2 and CRP were up-regulated. Further, in a mouse group that was pretreated with SeMet (selenomethionine) and showed a protective effect against the development of colorectal cancer, the expressions of PHB and PNP decreased compared to when SeMet (selenomethionine) alone was administered, but were up-regulated compared to a control group, and the expressions of ANXA2 and CRP decreased when colorectal cancer developed, but were up-regulated compared to a control group, suggesting that the four markers are all up-regulated when the development of colorectal cancer is inhibited by SeMet (selenomethionine). In addition, it was shown that the expression of 8-OHdG (8-hydroxy-2′-deoxyguanosine) that is an oxidative stress marker is regulated in a pattern similar to those of ANXA2 and CRP, suggesting that the oxidative stress marker 8-OHdG (8-hydroxy-2′-deoxyguanosine) is closely related to the expressions of PHB, PNP, ANXA2 and CRP.

Thus, when the expression levels of PHB, PNP, ANXA2 and CRP of the present invention are analyzed in combination, whether SeMet (selenomethionine) is to be administered can be determined and the development of colorectal cancer and the colorectal cancer inhibitory effect of SeMed can be monitored. Thus, these markers can be easily used for observation of prognosis after administration of SeMet (selenomethionine).

Hereinafter, the present invention will be described in further detail with reference to examples. It is to be understood, however, that these examples are for illustrative purposes only and are intended to limit the scope of the present invention. The examples of the present invention are provided in order to more completely explain the present invention to those skilled in the art.

### EXAMPLE 1

### Examination of Effect of SeMet (Selenomethionine) Administration on Decrease in AOM-DSS-Induced Polyps in Colorectal Cancer-Induced Mice

In order to examine the chemopreventive activity of SeMet (selenomethionine) against the development of colorectal cancer, the frequency and size of colon polyps in an inflammation-related colorectal cancer-induced mouse model according to the intake of SeMet (selenomethionine) were examined.

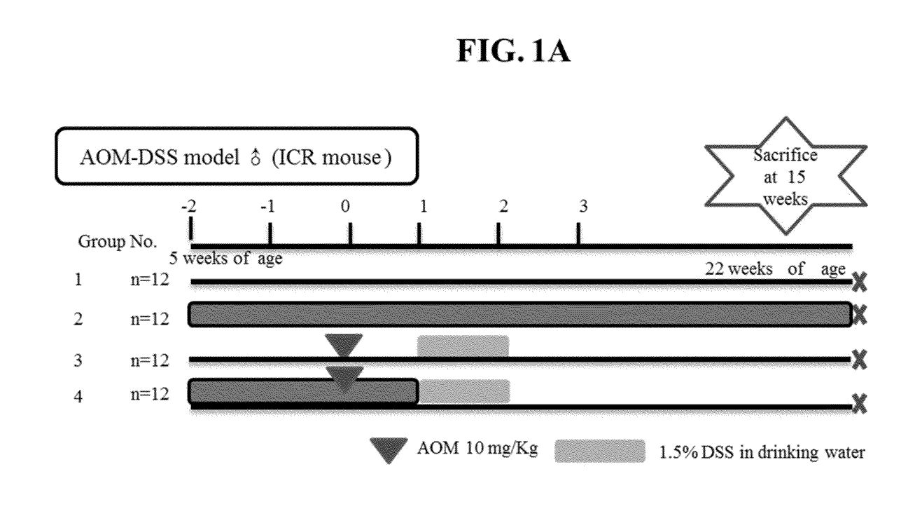

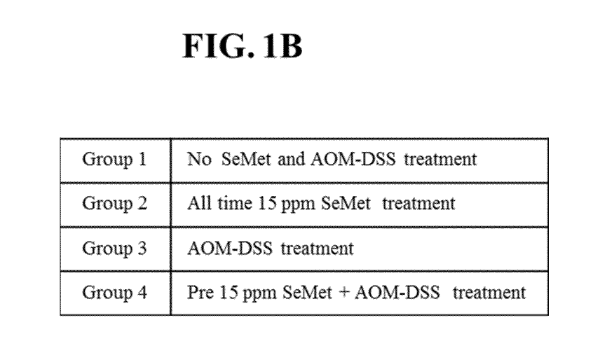

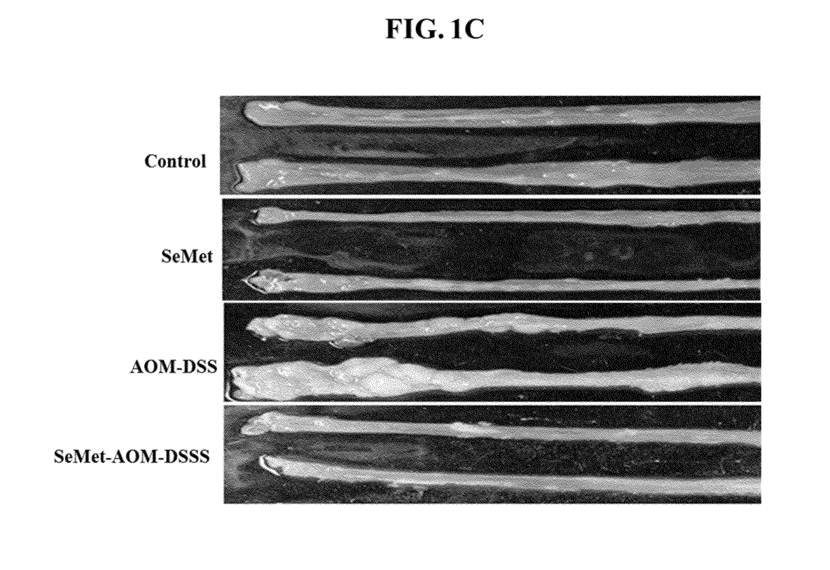

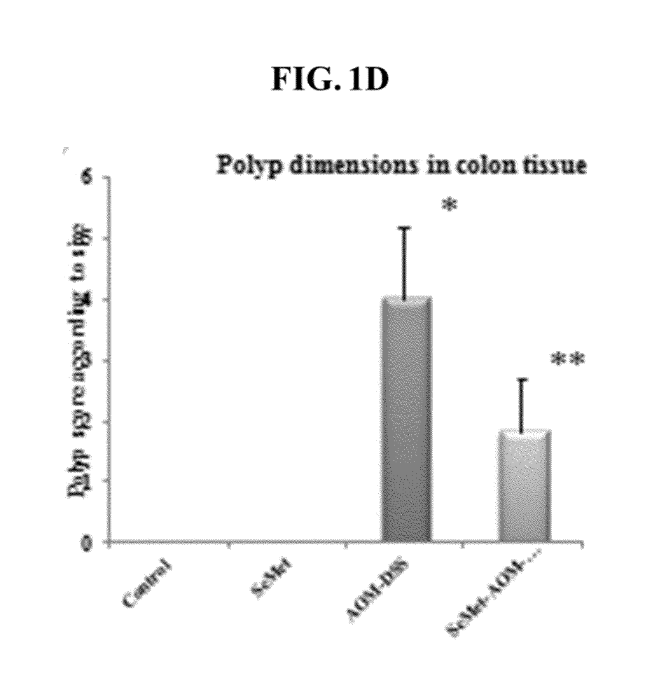

Specifically, an experiment was performed using forty eight 5-week-old ICR male mice (Lab Animal, Korea) divided into the following groups: group 1: treated with neither SeMet (selenomethionine) nor AOM-DSS; group 2: treated with 15 ppm SeMet (selenomethionine) (Pharma Se Inc, USA); group 3: treated with AOM-DSS; and group 4: pretreated with 15 ppm SeMet (selenomethionine) and then treated with AOM-DSS (FIGS. 1A and 1B).

AOM (azoxymethane) (Sigma-Aldrich Co, USA) that is a colorectal cancer-inducing substance was injected intraperitoneally (i.p.) into the mice at a dose of 10 mg/kg, and 1.5% (w/v) of dextran sodium sulfate (DSS) (MP Biomedicals, LLC, USA) that is a colitis-inducing substance was allowed to drink for one week after injection of AOM.

The mice of the four groups were euthanized with CO₂ gas when reached 22 weeks of age, and the colons were extracted and observed. In addition, the production of polyps in the colons was scored at a five-point scale as shown in Table 1 below for each size.

TABLE 1

| Polyp diameter (cm) | Score |
|---|---|
| 5 | 5 |
| 3 | 3 |
| 1 | 2 |
| 0.5 | 1 |

As a result, it could be seen that group 2 treated with 15 ppm of SeMet (selenomethionine) everyday was similar to group 1 (control group), suggesting that selenomethionine shows no toxicity, and polyps were more frequently found in group 3 treated with AOM-DSS. In addition, it could be seen that polyps in group 4, pretreated with SeMet (selenomethionine) and treated with AOM-DSS, significantly decreased compared to those in group 3 (FIGS. 1C and 1D). Thus, it can be seen that SeMet (selenomethionine) inhibits colorectal cancer.

### EXAMPLE 2

### Histopathological Observation of AOM-DSS-Induced Colorectal Cancer

Each of the colons extracted from the mice in Example 1 was fixed in 10% formalin, and then embedded in paraffin to make FFPE (paraffin-embedded) samples. Each of the FFPE samples was sectioned to a thickness of 10 μm and mounted on micro-slides (MUTO-GLASS, Japan), followed by drying at 37° C. overnight. Then, the paraffin sections were deparaffinized with xylene and concentration gradient alcohol. The deparaffinized tissue sections were stained with hematoxylin and eosin (H & E) (Sigma Aldrich) and an antibody (MOG-100P, JaICA) of 8-OHdG (8-hydroxy-2′-deoxyguanosine) known as an oxidative stress marker. The stained tissues were observed with an optical microscope (NIKON ECLIPSE 50i, Nikon).

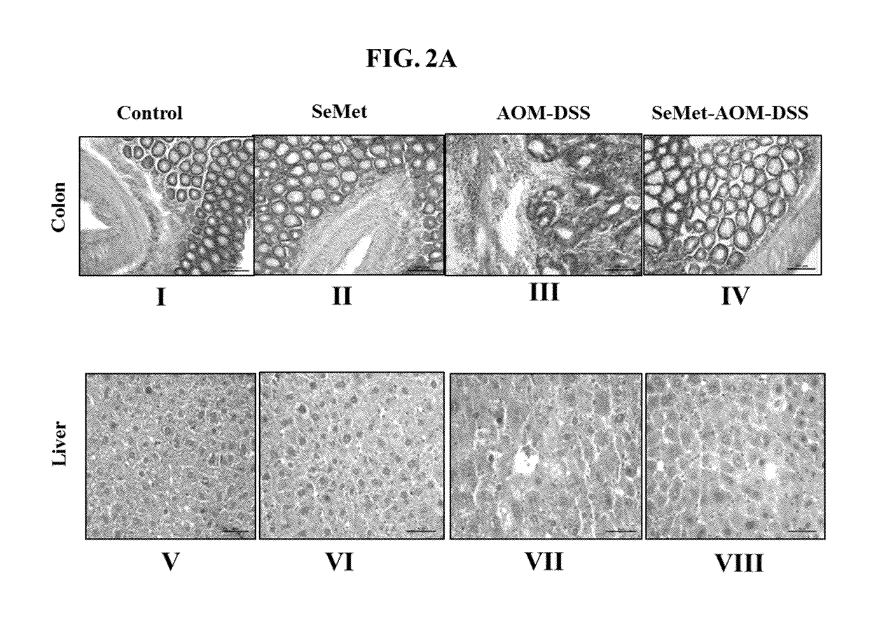

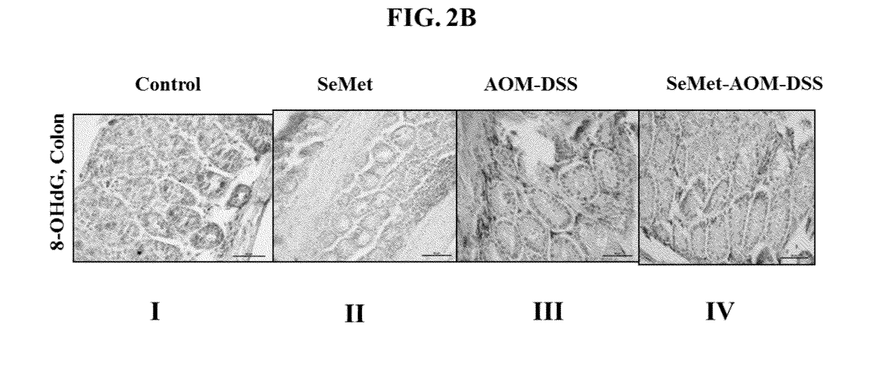

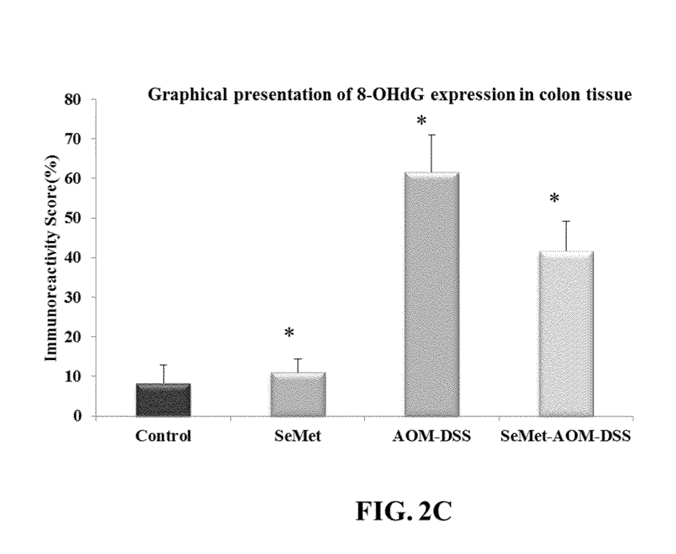

As a result, it could be seen that group 2 treated with 15 ppm of SeMet (selenomethionine) everyday was similar to group 1 (control group), and group 4 pretreated with SeMet (selenomethionine) before treatment with AOM-DSS showed decreases in dysplasia and neoplastic lesions compared to group 3 treated with AOM-DSS alone (FIG. 2A). Thus, it can be seen that SeMet (selenomethionine) inhibits colorectal cancer.

In addition, the results of staining of the oxidative stress marker 8-OHdG indicated that 8-OHdG increased in the group treated with AOM-DSS and that 8-OHdG in the group, pretreated with SeMet (selenomethionine) and treated with AOM-DSS, decreased compared to that in the group treated with AOM-DSS (FIGS. 2B and 2).

### EXAMPLE 3

### Investigation of Molecular Target of SeMet (Selenomethionine) having Chemopreventive Activity against Colorectal Cancer

3-1: 2-DE (2-Dimensional Electrophoresis) Analysis

In order to investigate the molecular target of SeMet (selenomethionine) having chemopreventive activity against colorectal cancer, the colon tissue samples obtained from the mice of groups 1 to 4 in Example 1 were analyzed using a 2-DE (2-dimensional electrophoresis) method.

Specifically, the colon tissues (excluding polyps) obtained from group 1 treated neither with SeMet (selenomethionine) nor AOM-DSS, group 2 treated with 15 ppm SeMet (selenomethionine) (Pharma Se Inc, USA), group 3 treated with AOM-DSS and group 4 treated with AOM-DSS after pretreatment with 15 ppm SeMet (selenomethionine) were washed with homogenization buffer A (50 mM Tris-HCl (pH7.5), 2 mM EDTA, 150 mM NaCl and 0.5 mM DTT) and then cut to small pieces. The pieces were homogenized in buffer (50 mM Tris-HCl (pH 7.5), 0.25 M sucrose, 5 mM magnesium acetate, 0.2 mM EDTA and 0.5 mM DTT) supplemented with Halt™ protease inhibitor cocktail (Thermo Fisher Scientific, Rockford, Ill.) on ice using a grinding kit (GE Healthcare Life Science, Uppsala, Sweden). Then, the solution was centrifuged at 13,000 rpm at 4° C. for 30 minutes, and 10% trichloroacetic acid was added to the supernatant to precipitate proteins. The collected precipitate was dissolved in rehydration buffer (8 M urea, 2% CHAPS, 50 mM DTT and 0.2% IPG buffer), and then, in order to perform 2D gel electrophoresis, the concentration of the proteins was adjusted with a BCA protein analysis kit (Thermo Fisher Scientific), and 200 μg of each protein was separated with Immobiline Dry Strip (pH 4-7, 18 cm, GE healthcare). 2D separation was performed on 12% acrylamide gel in Ettan Dalt II system (10 mA/gel; 1 hr, 40 mA/gel; >6 hr) (GE Healthcare Life Science, Uppsala, Sweden) for 7 hours. Then, the gel having proteins separated thereon stained using silver staining technology, after which the image of the gel was analyzed using Progenesis SameSpots software (version. 4.1, Nonlinear Dynamics, Newcastle, UK), and spots on the gel were detected. In analysis of the gel image, the gel was automatically aligned by measurement of alignment vectors using an analysis wizard, and master images of the experimental groups were made using Progenesis SameSpots software. The master images were used to normalize and quantify the spot volume and to analyze the proteins showing a difference in expression between the groups.

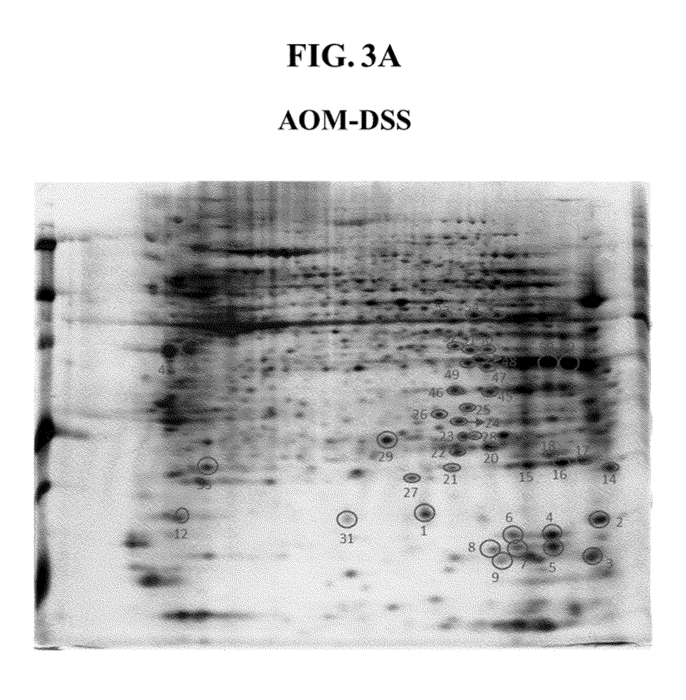

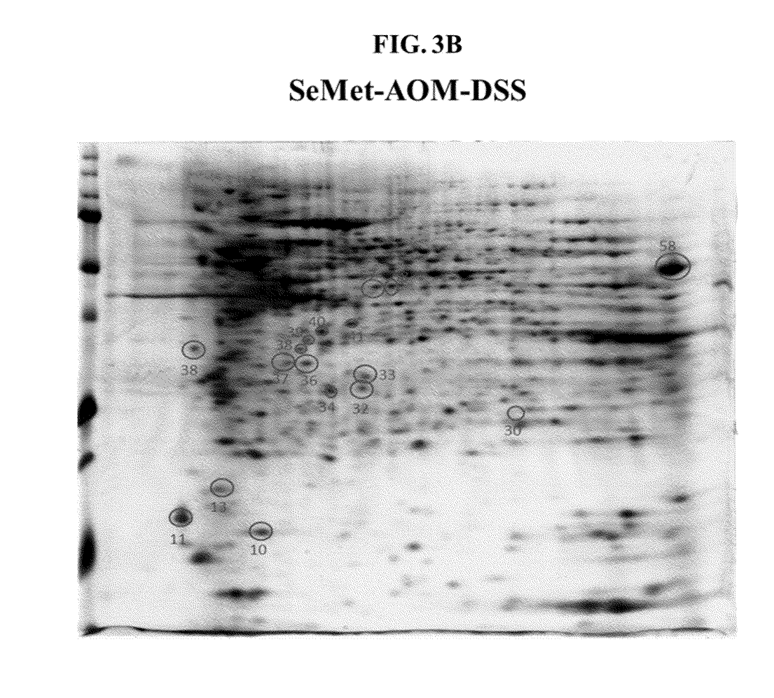

As a result, 76 protein spots were identified which showed a difference in expression between group 3 treated with AOM-DSS along and group 4 pretreated with SeMet (selenomethionine) before treatment with AOM-DSS (FIG. 3).

3-2: Nano-HPLC-ESI-QIT-MS Analysis

In order to investigate the molecular target of SeMet (selenomethionine) having chemopreventive activity against colorectal cancer, the colon tissue samples obtained from groups 1 to 4 in Example 1 were analyzed by mass spectrometry.

Specifically, 76 protein spots that showed a change in expression were cut from the 2D gel used in Example 3-1 and comprising the samples of groups 1 to 4. The cut spots were treated with trypsin, and protein identification was performed using a nano LC/MS system composed of a Surveyor HPLC system (Thermo Scientific, Waltham, Mass.) equipped with a nano-ESI source and an electrospray ionization (ESI)-quadrupole ion trap (QIT) mass spectrometer (LCQ Deca XP-Plus, Thermo Finnigan, San Jose, Calif., USA). In order to desalt and concentrate 10 μl of trypsin peptides, the peptide was loaded into a C18 trap column (i.d. 300 μm, length 5 mm, particle size 5 μm; LC Packings, Amsterdam, Netherlands) through an auto sampler at a flow rate of 20 μl/min. Then, the trapped peptides were allowed to flow backward and separated in a C18 reversed-phase capillary column (75 μm silica tube, length 150 mm, particle size 5 μm). The pump flow rate was split 1:100 for a column flow rate of 150 μl/min. Mobile phase A was a solution of a mixture of 0.5% acetic acid and 0.02% formic acid in water, and mobile phase B was a solution of a mixture of 0.5% acetic acid and 0.02% formic acid in 80% acetonitrile. The samples were injected into the column and eluted by mobile phase B at a concentration gradient of 5-5 20 50 60 80 100% for 0-15-18-50-55-60-62 minutes, respectively. MS and MS/MS spectra were obtained using a capillary tube (temperature: 220° C., ESI voltage: 2.5 kV, and collision energy: 35%). Data-dependent peak selection was most frequently used in the mass spectra. The MS/MS mass peaks were analyzed using SEQUEST software (version 3.3.1, Theremo Finnigan, San Jose, Calif.). SEQUEST was used for the identification of proteins using the IPI database. The results of the analysis were filtered using the following parameters: a mass tolerance of 2.0 Da for the precursor ion and 1.0 Da for the fragment ions, one missed cleavage per peptide was allowed, and modifications of proteins were not taken into account. The validity of peptide/spectrum matches was assessed using the SEQUEST defined parameters, the cross-correlation score (Xcor), and the normalized difference in cross-correlation scores. Matched peptide sequences were required to pass the following filters for identification: 1) the uniqueness scores of the matches' normalized difference in cross-correlation scores were at least 0.1, and 2) minimum Xcor values ≧1.90, ≧2.20, ≧3.75 for singly, doubly, and triply charged ions, respectively. Thus, among the 76 proteins that showed a difference in expression between group 3 treated h AOM-DSS and group 4 pretreated with SeMet (selenomethionine) and treated with AOM-DSS, 30 proteins whose expression increased or decreased were identified (Table 2).

TABLE 2

| | Gene | | Spot | Expression in |
|---|---|---|---|---|
| Protein name | symbol | Protein ID | number | SeMet/AOM-DSS |
| Annexin 3 | Anxa3 | IPI00132722.8 | 40 | Increased |
| Annexin 7 | Anxa7 | IPI00114017.2 | 57 | Increased |
| Beta-actin | Actb | IPI00110850.1 | 39 | Increased |
| Eukaryotic translation initiation 5A | Eif5a | IPI00108125.4 | 10 | Increased |
| Inorganic pyrophosphatase 1 | Ppa1 | IPI00110684.1 | 38 | Increased |
| Isoform 1 of Isocitrate dehydrogenase | Idh3a | IPI00459725.2 | 41 | Increased |
| [NAD] subunit alpha | | | | |
| Prohibitin | Phb | IPI00133440.1 | 34 | Increased |
| Proteasome activator complex subunit 1 | Psme1 | IPI00124223.1 | 32 | Increased |
| Purine nucleoside phosphorylase | Pnp | IPI00315452.5 | 33 | Increased |
| Aldose reductase | Akr1b3 | IPI00223757.4 | 49 | Decreased |
| Alcohol dehydrogenase | Akr1a4 | IPI00466128.3 | 50 | Decreased |
| Annexin 1 | Anxa1 | IPI00230395.5 | 51 | Decreased |
| Annexin 2 | Anxa2 | IPI00468203.3 | 48 | Decreased |
| Cofilin 1 | Cfl1 | IPI00407543.2 | 4 | Decreased |
| Cofilin 2 | Cfl2 | IPI00266188.6 | 4 | Decreased |
| C-reactive protein | Crp1 | IPI00314936.1 | 14 | Decreased |
| Destrin | Dstn | IPI00127942.4 | 5 | Decreased |
| Glutathione transferase omega 1 | Gsto1 | IPI00114285.1 | 25 | Decreased |
| Hypoxanthine-guanine | Hprt1 | IPI00284806.8 | 29 | Decreased |
| phosphoribosyltransferase 1 | | | | |
| Isoform 1 of Tropomyosin alpha-1 | Tpm1 | IPI00123316.1 | 43 | Decreased |
| chain | | | | |
| L-lactate dehydrogenase A chain | Ldha | IPI00319994.6 | 47 | Decreased |
| Nucleoside diphosphate kinase B | Nme2 | IPI00127417.1 | 8 | Decreased |
| Peroxiredoxin 1 | Prdx1 | IPI00121788.1 | 16, 21 | Decreased |
| Peroxiredoxin 4 | Prdx4 | IPI00116254.1 | 16, 21 | Decreased |
| Phosphoglycerate mutase 2 | Pgam2 | IPI00230706.5 | 24 | Decreased |
| Proteasome subunit beta type 1 | Psmb1 | IPI00113845.1 | 18 | Decreased |
| precursor | | | | |
| S-formylglutathione hydrolase | Esd | IPI00109142.4 | 46 | Decreased |
| Triosephosphate isomerase 1 | Tpi1 | IPI00467833.5 | 19, 23 | Decreased |
| Transaldolase | Taldo1 | IPI00124692.1 | 52 | Decreased |
| Ubiquinol cytochrome c reductase 1 | Uqcrfs1 | IPI00133240.1 | 20 | Decreased |

### EXAMPLE 4

### Analysis of Pathways of Proteins Using Pathway Studio 8 Software

In order to find pathways that regulate SeMet (selenomethionine)-mediated protective activity in colorectal cancer, the networks of the 30 proteins identified in Example 3 were analyzed using Pathway Studio 8 software.

Specifically, Pathway Studio 8 software (Ariadne Genomics, Rockville, Md., USA) was used to examine the functional interactions and possible pathways of the 30 proteins that showed a change in expression in colorectal cancer when pretreated with SeMet (selenomethionine).

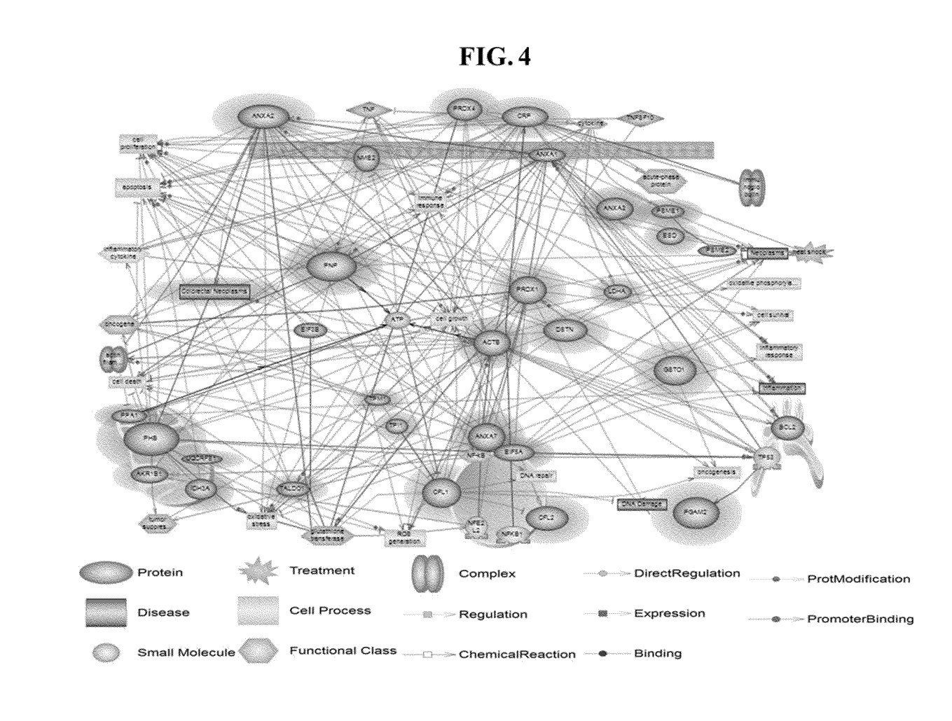

As a result, it was found that the following 27 proteins among the 30 proteins were related to each other: prohibitin (PHB), purine nucleoside phophorylase (PNP), isocitratrate dehydrogenase 3 alpha (IDH3A), eukaryotic translation initiation 5A (EIF5A), proteasome activator complex subunit 1 (PSME1), inorganic pyrophosphatase 1 (PPA1), beta actin (ACTB), annexin 7 (ANXA7) and annexin 3 (ANXA3), which were up-regulated by SeMet (selenomethionine) in the AOM-DSS mice treated with SeMet (selenomethionine), annexin 1(ANXA1), annexin A2 (ANXA2), cofilin 1 (CFL1), cofilin 2 (CFL2), c-reactive protein (CRP1), destrin (DSTN), glutathione transferase omega 1 (GSTO1), hypoxanthineguanine phosphoribosyltransferase 1 (HPRT1), tropomyosin alpha-1 chain (TPM1), L-lactate dehydrogenase A chain (LDHA), nucleoside diphosphate kinase B (NME2), peroxiredoxin 1 (PRDX1), peroxiredoxin 4 (PRDX2), phosphoglycerate mutase 2 (PGAM2), Sformylglutathione hydrolase (ESD), triosephosphate isomerase 1 (TPI1), transaldolase (TALDO1) and ubiquinol cytochrome c reductase 1 (UQCRFS1), which were down-regulated by SeMet (selenomethionine) in the AOM-DSS mice treated with SeMet (selenomethionine). In addition, it could be seen that the above proteins show changes in their expression, because SetMet and AOM-DSS influence cell proliferation, apoptosis, cell survival, cell growth, necrosis, ROS production, oxidative stress, inflammation, immune response and cellular positions, which are related to other small molecular substances, transcription factors, ligands and the like (FIG. 4).

In addition, the pathways of the proteins were analyzed, and as a result, the up-regulated proteins prohibitin (PHB) and purine nucleoside phosphorylase (PNP) and the down-regulated proteins annexin A2 (ANXA2) and C-reactive protein (CRP), which play the most important role in the pathways, were selected and determined as markers.

### EXAMPLE 5

### Identification of Markers Specific to Colorectal Cancer Preventive Activity of SeMet (Selenomethionine)

5-1: Immunohistochemical Analysis of Markers Specific to Colorectal Cancer Preventive Activity of SeMet (Selenomethionine) in Colorectal Cancer

Immunohistochemical analysis of the PHB, PNP, ANXA2 and CRP markers determined in Example 4 for the colon tissue samples obtained from groups 1 to 4 in Example 1 was performed.

Specifically, the colon paraffin sections obtained from the mice of groups 1 to 4 in Example 2 were deparaffinized and rehydrated. In addition, endogenous peroxidases were quenched with methanol containing 0.3% H₂O₂ for 20 minutes. The sections were incubated with the primary antibodies anti-prohibitin (H-80) (sc-28259, Santa Cruz Biotechnology), anti-PNP (sc-135163, Santa Cruz Biotechnology), anti-CRP (H-90) (sc-30047, Santa Cruz Biotechnology), anti-annexin II (H-50) (sc-9061, Santa Cruz Biotechnology) and anti-8-OhdG (MOG-100P, JaICA) at 4° C. overnight. Then, the sections were incubated with biotin-conjugated secondary antibodies corresponding to the primary antibodies for 30 minutes, after which the sections were washed with PBS and incubated with streptavidin horseradish peroxidase (Vector Labs) for 30 minutes. The sections were washed with PBS, and then incubated with a DAB (3,3′-diaminobenzidine) substrate solution containing 1.8×10⁻³% (v/v) of H₂O₂ for 10 minutes. After incubation, the sections were washed twice with PBS and stained with Gill's hematoxylin. The degree of staining of each of the markers in tumor cells developed in the stained tissues of each group was measured according to the method described in “Charafe-Jauffret, E., et al. (2004). J Pathol, 202, 265-73”.

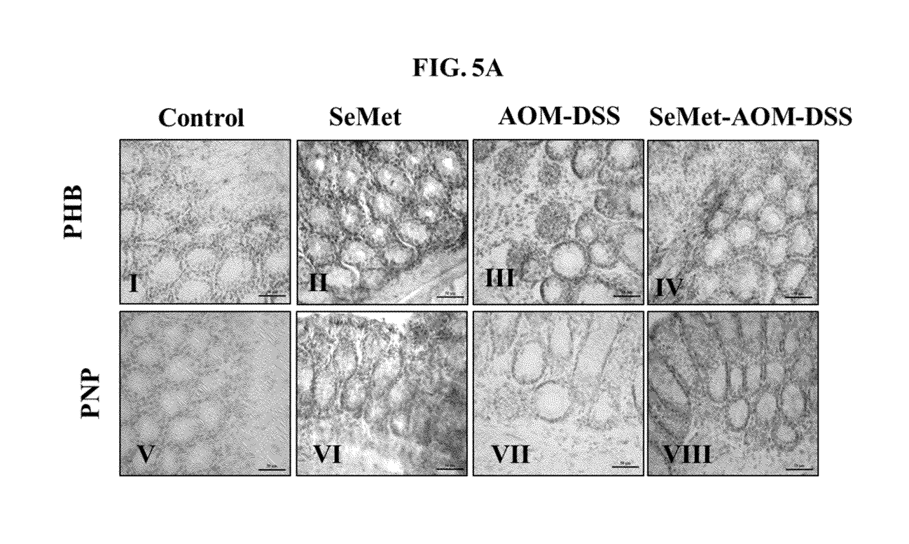

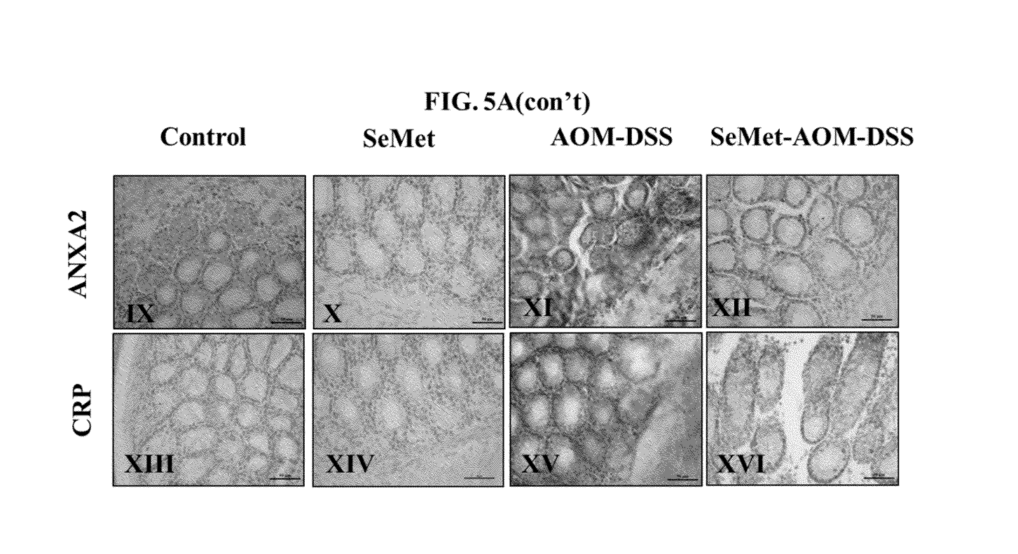

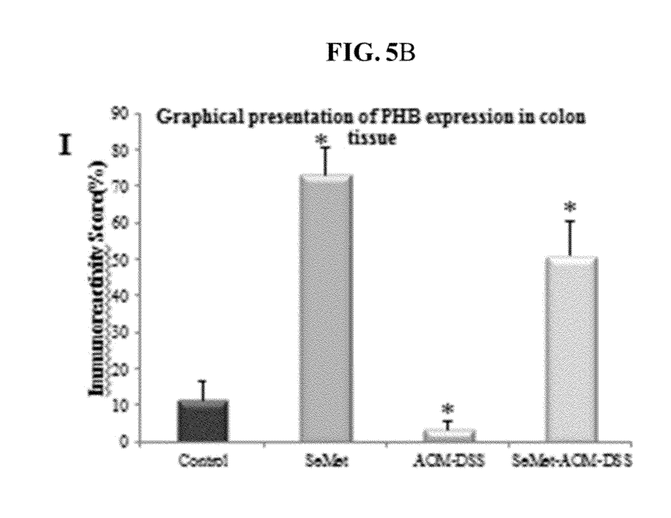

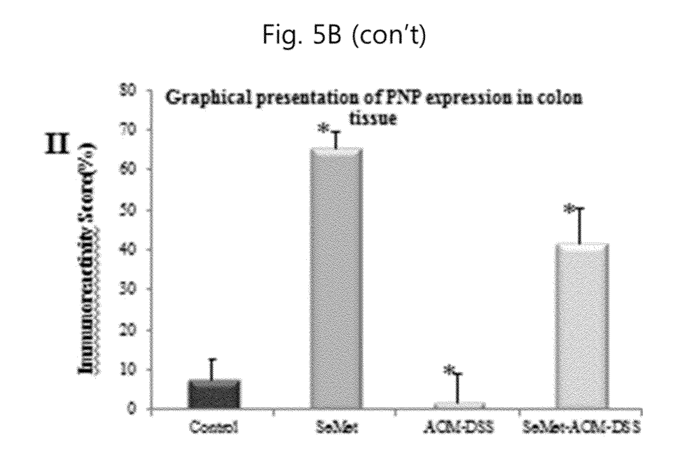

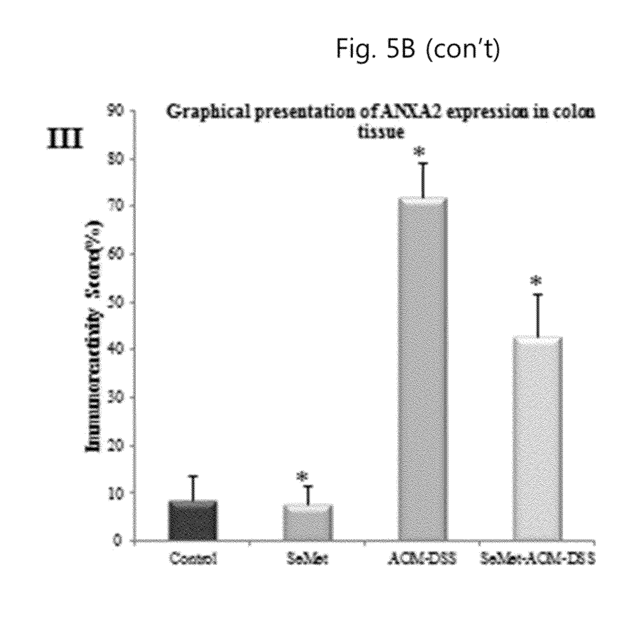

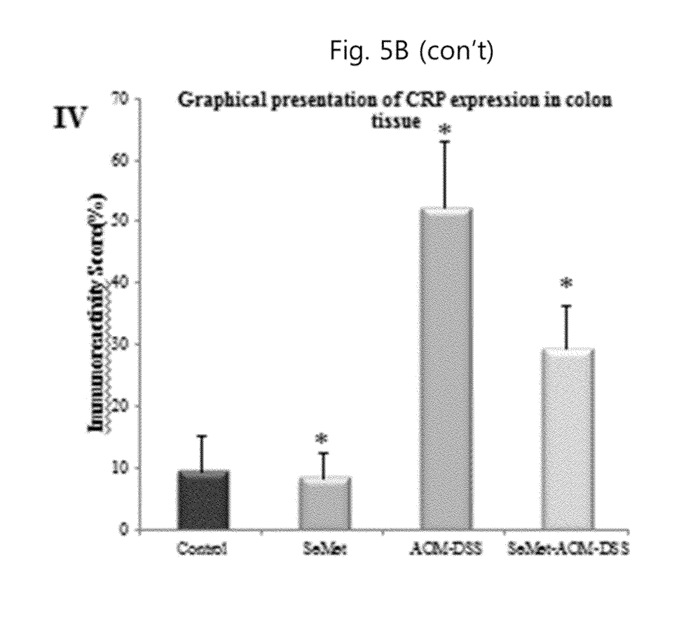

As a result, it could be seen that the expressions of PHB and PNP were increased by administration of SeMet (selenomethionine), and these markers were not substantially expressed in the tissues having colorectal cancer induced by AOM-DSS, and the expressions thereof increased again in group 4 in which the development of colorectal cancer was prevented by SeMet (selenomethionine). In addition, it was observed that the expressions of ANXA2 and CRP increased upon the development of colorectal cancer, but decreased upon pretreatment with SeMet (selenomethionine) (FIG. 5).

Thus, whether SeMet (selenomethionine) is to be administered can be determined by an increase in the expressions of PHB and PNP, and the development of colorectal cancer can be detected by an increase in the expressions of ANXA2 and CRP. In addition, when the expression levels of the four markers are analyzed in combination, whether SeMet (selenomethionine) is to be administered to prevent colorectal cancer can be determined (increases in the expressions of PHB and PNP, and no change in the expressions of ANXA2 and CRP), the development of colorectal cancer can be detected (increases in the expressions of ANXA2 and CRP, and no change in the expressions of PHB and PNP), and the inhibitory effect of SeMet (selenomethionine) against the development of colorectal cancer can be monitored (increases in the expressions of PHB, PNP, ANXA2 and CRP).

5-2: Analysis of Expressions of Markers Specific to Colorectal Cancer Preventive Activity of SeMet (Selenomethionine)

The expression levels of the PHB, PNP, ANXA2 and CRP markers in the colon tissue samples obtained from groups 1 to 4 in Example 1 were examined by Western blot analysis.

Specifically, from the colon tissues obtained from groups 1 to 4 in Example 1, proteins were extracted using the PRO-PREP™ Protein Extraction kit (cat. no. 17081) and quantified by the BCA method. 500 μg of the quantified proteins were loaded on gel, and then electrophoreased using running buffer (10×Tris/Glycine/SDS) (cat. no. 161-0732; Hercules, Calif., USA) and transfer buffer (25 mM Tris, 192 mM glycine and 10% methanol). After electrophoresis, the protein were transferred to a membrane, and then analyzed using anti-prohibitin (H-80) (sc-28259, Santa Cruz Biotechnology), anti-PNP (sc-135163, Santa Cruz Biotechnology), anti-CRP (H-90) (sc-30047, Santa Cruz Biotechnology) and anti-annexin II (H-50) (sc-9061, Santa Cruz Biotechnology) antibodies, and beta-actin antibody (Sigma, Catalog Number A3854) as a control.

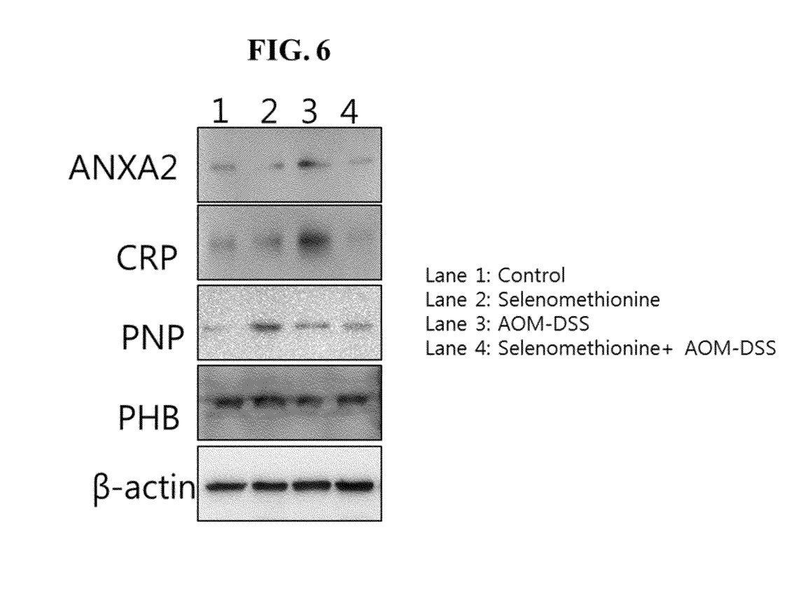

As a result, the proteins showed expression patterns similar to those in Example 5-1 (FIG. 6).

### EXAMPLE 6

### Analysis of Pathways of Markers Specific to Colorectal Cancer Preventive Activity of SeMet (Selenomethionine) Using Pathway Studio 8 Software

In order to examine the functional interactions and possible pathways of 8-OHdG whose expression was increased by the development of colorectal cancer and decreased by pretreatment with SeMet (selenomethionine) in Example 2 and the PHB, PNP, ANXA2 and CRP markers whose expressions were analyzed in Example 5-1, the pathways of the markers were analyzed using Pathway Studio 8 software (Ariadne Genomics, Rockville, Md., USA).

As a result, it could be seen that the PHB, PNP, ANXA2 and CRP markers are directly or indirectly related to 8-OHdG and colorectal cancer through apoptosis, oxidative stress and cytoplasm division (FIG. 7).

As described above, when the expressions of the biomarkers according to the present invention are measured and the expression levels thereof are analyzed in combination, whether SeMet (selenomethionine) is to be administered to prevent colorectal cancer can be determined and the development of colorectal cancer and the inhibitory effect of SeMet (selenomethionine) against the development of colorectal cancer can be monitored. Thus, these markers can be effectively used to observe the colorectal cancer inhibitory effect of SeMet (selenomethionine) and the prognosis of colorectal cancer resulting from the intake of SeMet (selenomethionine).

What is claimed is:

1. A composition for detecting the colorectal cancer inhibitory effect of SeMet (selenomethionine), the composition comprising agents for measuring the expression level of PHB (prohibitin) or PNP (purine nucleoside phosphorylase) and the expression level of ANXA2 (annexin A2) or CRP (C-reactive protein).

2. The composition of claim 1, wherein the agents for measuring the expression level are probes, primers, antibodies or aptamers.

3. The composition of claim 1, wherein PHB (prohibitin) has an amino acid sequence set forth in SEQ ID NO: 1, PNP (purine nucleoside phosphorylase) has an amino acid sequence set forth in SEQ ID NO: 2, ANXA2 (annexin A2) has an amino acid sequence set forth in SEQ ID NO: 3, and CRP (C-reactive protein) has an amino acid sequence set forth in SEQ ID NO: 4.

4. A kit for detecting the colorectal cancer inhibitory effect of SeMet (selenomethionine), the kit comprising the composition of claim 1.

5. A method for providing information required to monitor the colorectal cancer inhibitory effect of SeMet (selenomethionine), the method comprising a step of measuring the expression of at least one protein selected from the group consisting of PHB (prohibitin), PNP (purine nucleoside phosphorylase), ANXA2 (annexin A2) and CRP (C-reactive protein) in a sample separated from a subject.

6. The method of claim 5, wherein the sample is at least one selected from the group consisting of tissue, phlegm, blood, plasma and urine.

7. The method of claim 6, wherein the tissue is colon tissue or a colon cell separated therefrom.

8. The composition of claim 5, wherein PHB (prohibitin) has an amino acid sequence set forth in SEQ ID NO: 1, PNP (purine nucleoside phosphorylase) has an amino acid sequence set forth in SEQ ID NO: 2, ANXA2 (annexin A2) has an amino acid sequence set forth in SEQ ID NO: 3, and CRP (C-reactive protein) has an amino acid sequence set forth in SEQ ID NO: 4.

9. The composition of claim 5, wherein the expression of PHB (prohibitin) or PNP (purine nucleoside phosphorylase) is increased by administration of SeMet (selenomethionine) and decreased by development of colorectal cancer.

10. The composition of claim 5, wherein the expression of ANXA2(annexin A2) or CRP (C-reactive protein) is increased by development of colorectal cancer and decreased by administration of SeMet (selenomethionine).

11. The composition of claim 5, wherein, when the expression of PHB (prohibitin) or PNP (purine nucleoside phosphorylase) together with the expression of ANXA2 (annexin A2) or CRP (C-reactive protein) increases, SeMet (selenomethionine) is determined to have a tumor inhibitory or preventive effect.


SEQUENCE LISTING (4 sequences)

SEQ ID NO: 1 - protein, 272 aa, Mus musculus
MAAKVFESIGKFGLALAVAGGVVNSALYNVDAGHRAVIFDRFRGVQDIVVGEGTHFLIPWVQKPIIFDCRSRPRNVPVITGSKDLQNVNITLRILFRPVASQLPRIYTSIGEDYDERVLPSITTEILKSVVARFDAGELITQRELVSRQVSDDLTERAATFGLILDDVSLTHLTFGKEFTEAVEAKQVAQQEAERARFVVEKAEQQKKAAIISAEGDSKAAELIANSLATAGDGLIELRKLEAAEDIAYQLSRSRNITYLPAGQSVLLQLPQ

SEQ ID NO: 2 - protein, 289 aa, Mus musculus
MENEFTYEDYETTAKWLLQHTEYRPQVAVICGSGLGGLTAHLKEAQIFDYNEIPNFPQSTVQGHAGRLVFGLLNGRCCVMMQGRFHMYEGYSLSKVTFPVRVFHLLGVETLVVTNAAGGLNPNFEVGDIMLIRDHINLPGFCGQNPLRGPNDERFGVRFPAMSDAYDRDMRQKAFSAWKQMGEQRKLQEGTYVMLAGPNFETVAESRLLKMLGADAVGMSTVPEVIVARHCGLRVFGFSLITNKVVMDYENLEKANHMEVLDAGKAAAQTLERFVSILMESIPLPDRGS

SEQ ID NO: 3 - protein, 339 aa, Mus musculus
MSTVHEILCKLSLEGDHSTPPSAYGSVKPYTNFDAERDALNIETAVKTKGVDEVTIVNILTNRSNVQRQDIAFAYQRRTKKELPSALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARELYDAGVKRKGTDVPKWISIMTERSVCHLQKVFERYKSYSPYDMLESIKKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 4 - protein, 225 aa, Mus musculus
MEKLLWCLLIMISFSRTFGHEDMFKKAFVFPKESDTSYVSLEAESKKPLNTFTVCLHFYTALSTVRSFSVFSYATKKNSNDILIFWNKDKQYTFGVGGAEVRFMVSEIPEAPTHICASWESATGIVEFWIDGKAKVRKSLHKGYTVGPDASIILGQEQDSYGGDFDAKQSLVGDIGDVNMWDFVLSPEQINTVYVGGTLSPNVLNWRALNYKAQGDVFIKPQLWS